(19) United States
(12) Patent Application Publication
(10) Pub. No.: US 2008/0092908 A1
(43) Pub. Date: Apr. 24, 2008

(54) Orthopedic and Anti-Apnea Apparatus for Reposing or Sleeping

(76) Inventor: Fortunato Costa, Linda-a-Velha (PT)

Correspondence Address: COSTA, Fortunato, Av. 25 Abril, 22, 2-D, Linda-a-Velha, 2795-196, PT

(21) Appl. No.: 11/574,875

(22) PCT Filed: Sep. 8, 2005
(86) PCT No.: PCT/PT05/00013

§ 371 Date: Mar. 7, 2007

(30) Foreign Application Priority Data
Sep. 13, 2004 (PT) 103191

(51) Int. Cl. A61F 5/56

(52) U.S. Cl. 128/848

(57) ABSTRACT

The apparatus (4, 5) allows sleeping on the back without any height or declivity between the head and the bed maintaining horizontal orthopedic correct position for the vertebral bone structure and allowing the air to flow more naturally in the respiratory system, reducing the apnea problem and improving the sleep quality. The apparatus allows further that the head is elevated above the bed only when a human sleeps on his left side (1) or right side (3) and that when a person turns to his side his head will be elevated gradually.

### DEFINITION

Orthopedic and anti-apnea method and apparatus (4) or (5), characterized by being based in a device for reposing or sleeping where:

the middle part (8), without thickness or height, is used to accommodate the head when sleeping with the backside down, so with the belly up (2), allowing the head, the neck and the rest of the body to stay completely horizontal on the surface of the bed;

the left part (6) already has a significant thickness or height to compensate the sleeping turned over the left shoulder, because it is not possible for the head to rest naturally on the surface of the bed and, so, allowing the head (1) to be elevated relatively to the surface of the bed, when a person sleeps turned to the left side;

the right part (7) also with a significant thickness or height, similar to the left part (6), to compensate the sleeping turned over the right shoulder, because it is not possible for the head to rest naturally on the surface of the bed and, so, allowing the head (3) to be elevated relatively to the surface of the bed, when a person sleeps turned to the right side;

between the middle part (8) and the left part (6) exists a small declivity, similar and symmetric to the declivity between the middle part (8) and the right part (7) allowing that when a person turns the head to the right side (1), or to the left side (3), the head will be elevated gradually when rotating the body relatively to the longitudinal axe of the body, so, when reposing or sleeping: the head, the neck and the body do maintain the same relative positions, between them, similar to the ones that a human has when stays erect, or walks, in the vertical position.

Retrospective

To sleep represents almost half of our life time. To improve the quality of our sleep is, certainly, to contribute for a much healthier and longer life.

Several patents exist already related with pillows to sleep, but none of them is based in the principle that the fact of reposing or sleeping, does not mean that the head, the neck and the body should have relative positions, between them, different than to the ones that a human has when he stays erect, or walks, in the vertical position.

Invention Field

The present invention is related with several medical sciences: orthopedics, apnea, respiratory system, obesity, third age or oldness, the sleep, between many others, where the good sleeping and the well-being has an intrinsic impact.

So, the application of the invention will be eventually more applied in industries related with the medicine, the improvements of the quality of life and of the well-being of the human kind.

Evolution

The orthopedic and anti-apnea method and apparatus for reposing or sleeping (4) or (5), is based in the principle that the fact of reposing or sleeping, does not mean that the head, the neck and the body should have relative positions, between them, different than to the ones that a human has when he stays erect, or walks, in the vertical position. When sleeping with the backside down, so with the belly up (2), there is no need to have a height or declivity between the head and the bed to support the head; allowing the head, the neck and the rest of the body to stay completely horizontal on the surface of the bed; maintaining by this way an horizontal orthopedic correct position for the vertebral bone structure and allowing the air to flow more naturally in the respiratory system, reducing the apnea problem and improving the sleep quality.

On the other side, the orthopedic and anti-apnea method and apparatus is, also, based in the fact that the head needs to be elevated above the surface of the head only when a human sleeps turned to the left side (1) or to the right side (3) and, just to be able to compensate the fact that sleeping turned over one of the shoulders does not let the head to repose naturally on the surface of the bed; and so, allowing that when a person turns the head to the right side (1), or to the left side (3), the head will be elevated gradually when rotating the body relatively to the longitudinal axe of the body.

The orthopedic and anti-apnea apparatus (4) also can be portable and be used attached around the head like a band (5) or like a helmet.

In the case of the portable usage of the apparatus, around the head like a band (5) or like a helmet, with the fact of completely covering the two ears we can have sound isolation, or on the other side, we can listen stereophonic music or sounds, just by implementing small ear speakers in the right side (6) and left side (7) of the orthopedic and anti-apnea apparatus (4) or (5), without disturbing other persons near by.

In the case of the portable usage of the apparatus, around the head like a band (5) or like a helmet it can be a solution to avoid the asphyxia problems of the traditional pillows in children, old people and patients, by not allowing that the respiratory entrances, mouth and nose, contact directly with the surface of the bed, or cradle.

The orthopedic and anti-apnea apparatus (4) or (5) can also be made to be insufflated and allowing, by this way, that the user can also regulate the hardness and height, above the surface of the bed, that he pretends to have the head when reposing or sleeping, turned to the left side (1) or to the right side (3).

Finally, the orthopedic and anti-apnea apparatus (5) can, also, be equipped with visual screens, sensors, microphones and/or computers that allow the user to receive and to send computerized and multimedia information, while the user is reposing.

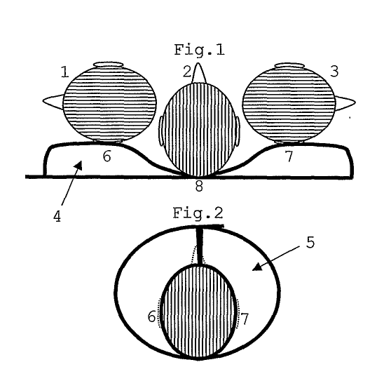

The FIG. 1, FIG. 1, represents the orthopedic and anti-apnea apparatus (4) and the way to be used in a normal horizontal mode, when a person reposes or sleeps on the surface of the bed: in the position (1) turned over the left shoulder; in the position (2) backside down, or with the belly up; or in the position (3) turned over the right shoulder.

The FIG. 2, FIG. 2, represents the possibility of the portable usage of the orthopedic and anti-apnea apparatus to repose or to sleep (5), attached around the head like a band (5) helmet.

1. Orthopedic and anti-apnea method and apparatus (4) or (5), characterized by being based in a device for reposing or sleeping where:
the middle part (8), without thickness or height, is used to accommodate the head when sleeping with the backside down, so with the belly up (2), allowing the head, the neck and the rest of the body to stay completely horizontal on the surface of the bed;
the left part (6) already has a significant thickness or height to compensate the sleeping turned over the left shoulder, because it is not possible for the head to rest naturally on the surface of the bed and, so, allowing the head (1) to be elevated relatively to the surface of the bed, when a person sleeps turned to the left side;
the right part (7) also with a significant thickness or height, similar to the left part (6), to compensate the sleeping turned over the right shoulder, because it is not possible for the head to rest naturally on the surface of the bed and, so, allowing the head (3) to be elevated relatively to the surface of the bed, when a person sleeps turned to the right side;
between the middle part (8) and the left part (6) exists a small declivity, similar and symmetric to the declivity between the middle part (8) and the right part (7) allowing that when a person turns the head to the right side (1), or to the left side (3), the head will be elevated gradually when rotating the body relatively to the longitudinal axe of the body, so, when reposing or sleeping: the head, the neck and the body do maintain the same relative positions, between them, similar to the ones that a human has when stays erect, or walks, in the vertical position.

2. Method and apparatus according to the claim 1, characterized by allowing that the apparatus also can be portable and be used attached around the head like a band (5) helmet.

3. Method and apparatus according to the claims 1 and 2, characterized by allowing, in the case of the portable usage of the apparatus, around the head like a band (5) or like a helmet, that the fact of completely covering the two ears we can have sound isolation, or on the other side, we can listen stereophonic music or sounds, just by implementing small ear speakers in the right side (6) and left side (7) of the orthopedic and anti-apnea apparatus (4) or (5).

4. Method and apparatus according to the claims from 1 to 3, characterized by—in the case of the portable usage of the apparatus, around the head like a band (5) or like a helmet—to be a solution to avoid asphyxia problems in children, old people and patients, not allowing that the respiratory entrances, mouth and nose, contact directly with the surface of the bed, or cradle.

5. Method and apparatus according to the claims from 1 to 4, characterized by the fact that the orthopedic and anti-apnea apparatus (4) or (5) can also be made to be insufflated and allowing, by this way, that the user can also regulate the hardness and height, above the surface of the bed, that he pretends to have the head when reposing or sleeping, turned to the left side (1) or to the right side (3).

6. Method and apparatus according to the claims from 1 to 5, characterized by the fact that the orthopedic and anti-apnea apparatus (5) can, also, be equipped with visual screens, sensors, microphones and/or computers that allow the user to receive and to send computerized and multimedia information, while the user is reposing.

7. The utilization of the method and apparatus according to the claims from 1 to 6, characterized by being used as an orthopedic apparatus.

8. The utilization of the method and apparatus according to the claims from 1 to 6, characterized by being used as an anti-apnea and respiratory improver apparatus.
